(19) United States
(12) Patent Application Publication
(10) Pub. No.: US 2024/0156515 A1
(43) Pub. Date: May 16, 2024

(54) SURGICAL CUTTING AND COAGULATION DEVICE

(71) Applicant: Donald J. Geisel, Clifton Park, NY (US)

(72) Inventor: Donald J. Geisel, Clifton Park, NY (US)

(21) Appl. No.: 18/505,457

(22) Filed: Nov. 9, 2023

Related U.S. Application Data
(60) Provisional application No. 63/475,504, filed on Nov. 15, 2022.
(60) Provisional application No. 63/384,413, filed on Nov. 19, 2022.

(51) Int. Cl. A61B 18/14; A61B 17/3211; A61B 18/12

(52) U.S. Cl. CPC A61B 18/1402; A61B 17/3211; A61B 18/1206; A61B 2018/00589

(57) ABSTRACT

A surgical cutting and coagulation device includes a cutting blade having a cutting edge and a metallic overlay affixed to the cutting blade configured to contact tissue during an incision of the tissue by the cutting edge of the cutting blade. The cutting blade is configured to cut or shear the tissue with the cutting edge while the metallic overlay touches and coagulates the tissue. The metallic overlay is powered by an RF generator. A method of cutting and coagulation includes powering the metallic overlay with the RF generator, cutting or shearing a tissue with the cutting edge, and during the cutting or shearing the tissue with the cutting edge, coagulating the tissue with the metallic overlay.

### RELATED MATTERS

This application claims the benefit of the earlier filing date of U.S. Provisional Patent Application No. 63/475,504 titled “Surgical cutting and coagulation device,” and the U.S. Provisional Patent Application No. 63/384,413, entitled “Surgical Cutting and Coagulation Device Rev. 1”, the entireties of which are each incorporated by reference.

### TECHNICAL FIELD

The present invention relates to surgical devices. More particularly, the present invention relates to an improved surgical cutting and/or coagulation device.

### BACKGROUND

The most common form of electrosurgery is the monopolar method where a generator supplies RF current to a surgical tip or pencil for tissue cutting and coagulation. With monopolar electrosurgery, current flows into the tissue and causes an incision by tissue vaporization and such current then flows to a patient pad and back via its return path to the generator. The monopolar method requires current flowing through a patient's tissue to the patient pad, which can be dangerous to vulnerable tissue of a patient.

Another form of RF electrosurgery is the bipolar method. With RF electrosurgery, RF current generally flows to forceps or a hemostat type clamp to supply current across a small region of tissue, generally clamped or squeezed in place. Here, the current only flows through the small area of tissue for coagulation purposes. RF bipolar electrosurgical methods are generally safer for the patient than monopolar methods because no stray monopolar current can flow through sensitive tissue areas such as peripheral nerves or implanted electronic devices.

Numerous unsuccessful attempts have been made to develop a bipolar method of cutting. For example, one unsuccessful bipolar method includes using two closely spaced cutting probes, one larger than the other, where current can flow through a small zone of tissue causing an incision by tissue vaporization. These unsuccessful approaches cause severe eschar to deposit on the probe surfaces. Another disadvantage of existing bipolar methods is that unwanted sparking occurs between the smallest probe member and patient tissue thus causing unwanted tissue jitter and electrical noise or EMI.

Thus, an improved surgical cutting and/or coagulating device would be well received in the art.

### SUMMARY

According to a described aspect, a surgical cutting and coagulation device comprises: a cutting blade having a cutting edge; and a metallic overlay affixed to the cutting blade configured to contact tissue during an incision of the tissue by the cutting edge of the cutting blade, where the cutting blade is configured to cut or shear the tissue with the cutting edge while the metallic overlay touches and coagulates the tissue, and where the metallic overlay is powered by an RF generator.

According to another described aspect, a surgical cutting and coagulation device comprises: a mechanical cutting member; and an electrical coagulation member with two or more metallic contacts, where the mechanical cutting member cuts or shears tissue with a knife edge while the electrical coagulation member touches and coagulates the tissue, and where the coagulation member is powered by a RF generator.

According to another described aspect, a method for cutting and coagulating comprises: providing a surgical cutting and coagulating device having a cutting blade with a cutting edge, and a metallic overlay affixed to the cutting blade configured to contact tissue during an incision of the tissue by the cutting edge of the cutting blade, where the cutting blade is configured to cut or shear the tissue with the cutting edge while the metallic overlay touches and coagulates the tissue, and where the metallic overlay is powered by an RF generator. The method includes providing the RF generator, powering the metallic overlay with the RF generator, cutting or shearing a tissue with the cutting edge, and during the cutting or shearing the tissue with the cutting edge, coagulating the tissue with the metallic overlay.

### BRIEF DESCRIPTION OF THE DRAWINGS

Some of the embodiments will be described in detail with references made to the following figures, wherein like designations denote like members, wherein:

FIG. 1 depicts a side view of a first side of a surgical cutting and coagulating device, in accordance with one embodiment.

FIG. 2A depicts a detailed view of the first side of a cutting blade of the surgical cutting and coagulating device of FIG. 1, in accordance with one embodiment.

FIG. 2B depicts a detailed view of a second side of the cutting blade of the surgical cutting and coagulating device of FIG. 1, in accordance with one embodiment.

FIG. 3 depicts a cross sectional view of the cutting blade of FIGS. 2A and 2B, taken at arrows 3-3, in accordance with one embodiment.

FIG. 4A depicts a perspective view of a first side of another surgical cutting and coagulating device, in accordance with one embodiment.

FIG. 4B depicts a perspective view of a second side of the surgical cutting and coagulating device of FIG. 4A, in accordance with one embodiment.

FIG. 5A depicts a perspective view of a first side of another surgical cutting and coagulating device, in accordance with one embodiment.

FIG. 5B depicts a perspective view of a second side of the surgical cutting and coagulating device of FIG. 5A, in accordance with one embodiment.

FIG. 6 depicts a schematic view of a bipolar electrosurgery system deploying the surgical cutting and coagulating device in use on a patient, in accordance with one embodiment.

FIG. 7 depicts a schematic view of a monopolar electrosurgery system, in accordance with one embodiment.

FIG. 8 depicts a method of surgical cutting and coagulating using a surgical cutting and coagulating device, in accordance with one embodiment.

### DETAILED DESCRIPTION

A detailed description of the hereinafter-described embodiments of the disclosed apparatus and method are presented herein by way of exemplification and not limitation with reference made to the Figures. Although certain embodiments are shown and described in detail, it should be understood that various changes and modifications might be made without departing from the scope of the appended claims. The scope of the present disclosure will in no way be limited to the number of constituting components, the materials thereof, the shapes thereof, colors thereof, the relative arrangement thereof, etc., and are disclosed simply as an example of embodiments of the present disclosure. A more complete understanding of the present embodiments and advantages thereof may be acquired by referring to the following description taken in conjunction with the accompanying drawings, in which like reference numbers indicate like features.

As a preface to the detailed description, it should be noted that, as used in this specification and the appended claims, the singular forms “a”, “an” and “the” include plural referents, unless the context clearly dictates otherwise.

The present disclosure provides for embodiments of devices and methods for safe cutting of a patient's tissue without the dangers of monopolar current flowing to a patient pad. Devices and associated methods are disclosed in which a cutting blade cuts by pure mechanical means via a scalpel or knife. Simultaneously to this mechanical cutting, coagulation is accomplished by bipolar RF energy. The coagulation effect may be adjustable by adjusting an RF generator output which is independent of the mechanical cutting action.

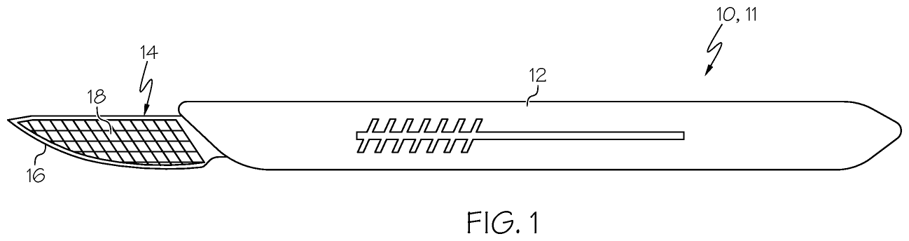

FIG. 1 depicts a side view of a first side 11 of a surgical cutting and coagulating device 10, in accordance with one embodiment. As shown, the surgical cutting and coagulating device 10 includes a handle 12, a cutting blade 14, and a cutting edge 16. The cutting blade 14 may be any mechanical cutting member, such as a scalpel, knife or the like. The cutting edge 16 may be a sharp knife edge or the like which is configured for mechanical cutting of tissue.

Located on the first side 11 of the cutting blade 14 is an overlay 18 which may be configured to contact tissue during an incision of the tissue by the cutting edge 16 of the cutting blade 14. The overlay 18 may be any form of an electrical coagulation member which is conductive of electricity and capable of being connected to an RF generator, as described herein below. The overlay 18 is shown as a structure which encompasses a large portion of the cutting blade 14. However, the overlay 18 may take any shape, including a thin strip, or the like. The overlay 18 may comprise a mesh of wires, rather than completely covering an area of the cutting blade 14. Whatever the embodiment, the overlay 18 may be configured to contact incised tissue as the cutting blade 14 cuts.

While the embodiment shown includes a particular handle and blade shape, various other blade shapes and handle shapes and sizes are contemplated. The cutting blade 14 may generally be made of a non-metallic material, such as a ceramic or zirconium. During surgery of a patient, the cutting blade 14 is configured to cut or shear the tissue of the patient with the cutting edge 16 while the overlay 18 touches and coagulates the tissue as described in more detail herein below.

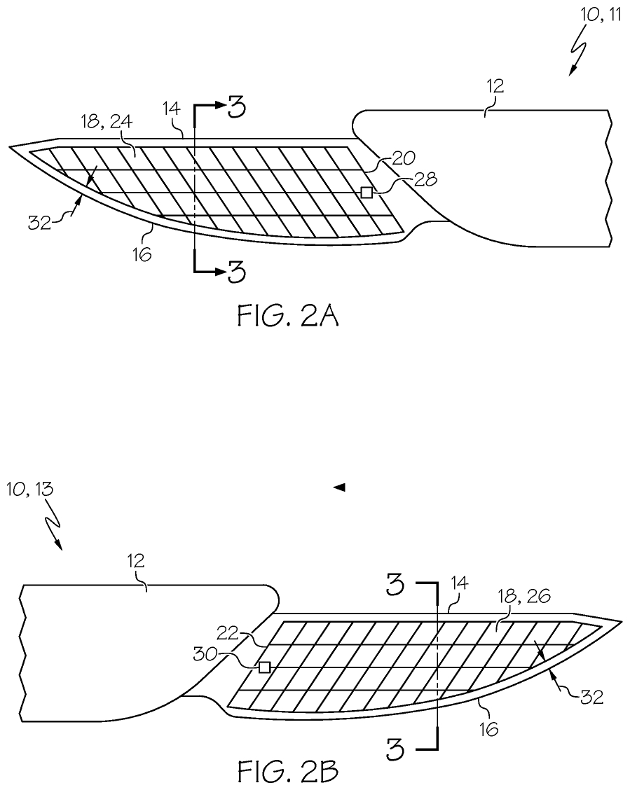

FIG. 2A depicts a detailed view of the first side 11 of a cutting blade 14 of the surgical cutting and coagulating device 10 of FIG. 1, in accordance with one embodiment. Likewise, FIG. 2B depicts a detailed view of a second opposite side 13 of the cutting blade 14 of the surgical cutting and coagulating device 10 of FIG. 1, in accordance with one embodiment. FIGS. 2A and 2B show that the cutting edge 16 is located on a shared perimeter of a first surface 20 (shown in FIG. 2A) and a second opposite surface 22 (shown in FIG. 2B) of the cutting blade 14. The overlay 18 includes a first portion 24 that is located on the first surface 20 of the cutting blade 14. Moreover, the overlay 18 includes a second portion 26 that is located on the second surface 22 of the cutting blade 14.

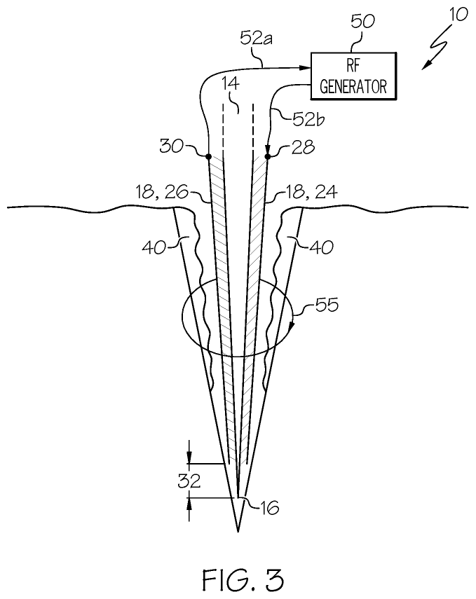

The first portion 24 of the overlay 18 includes a first electrical connection point 28 for connecting the first portion 24 of the overlay 18 to an RF (radio frequency) generator (shown in FIG. 3). Likewise, the second portion 26 of the overlay 18 includes a second electrical connection point 30 for connecting the second portion 26 of the overlay 18 to the RF generator.

Each of the first portion 24 and the second portion 26 of the overlay 18 is made of a metallic material that is bonded, affixed or coated to each of the respective first and second surfaces 20, 22 of the cutting blade 14. For example, each of the first portion 24 and the second portion 26 of the overlay 18 may be made of a thin sheet of metal affixed to the cutting blade 14. The thin sheet may be a thin silver sheet, for example. However, other metals are contemplated such as copper, gold, or the like. The thin sheet may have varying thicknesses, such as a thickness of about 0.01 inches or less. However, any appropriate thickness of the overlay 18 is contemplated. In still other embodiments, each of the first portion 24 and the second portion 26 of the overlay 18 is made of a conductive thick film or epoxy coated onto the cutting blade 14. The thick film or epoxy may be an electrically conductive coating, such as a coating made of silver, gold, carbon or the like.

As shown, a spacing 32 may be located between the cutting edge 16 and the overlay 18. The spacing 32 may span the length of the cutting edge 16 and may be located on each of the first surface 20 and the second surface 22. The spacing 32 may be configured to provide sufficient space to allow for the cutting edge 16 to perform as a conventional cutting device during the incision and mechanically cut tissue. However, the spacing 32 is narrow enough to allow the first and second portions 24, 26 of the overlay 18 to contact the tissue during the incision, as shown in FIG. 3.

FIG. 3 depicts a cross sectional view of the cutting blade 14 of FIGS. 2A and 2B, taken at arrows 3-3, in accordance with one embodiment. As shown, the first and second portions 24, 26 of the overlay 18 is shown in contact with incised tissue 40. Further shown in FIG. 3 are wires 52a, 52b connected between the wire electrical connections 28, 30 of each of the first portion 24 and the second portion 26 of the overlay 18, and an RF generator 50. The wires 52a, 52b connect the overlay 18 to an RF generator 50, which acts as an energy source to provide energy to the overlay 18 during coagulation simultaneous with cutting.

As an incision is made by the cutting edge 16, RF current flows from the RF generator 50 to the first and second portions 24, 26 of the overlay 18, and through the patient tissue 40 via a current path 55. The current path 55 flows through the incised tissue 40. This contact between the first and second portions 24, 26 of the overlay 18 and the incised tissue 40 is configured to impart coagulation and retard the flow of blood. The RF generator 50 connection to the cutting blade 14 via the wires 52a, 52b provides a bipolar technique whereby only a small portion of localized tissue 40 is subject to the electrical current.

While not shown in FIG. 3, it is also possible to employ an insulating layer between the cutting blade 14 and the overlay 18, and particularly between each of the first and second portions 24, 26 of the overlay 18. In such an embodiment, it is possible that the cutting blade 14 is made from a metallic material, rather than ceramic or zirconium. The insulating layer may be, for example, a ceramic insulator, a nonconductive epoxy, a glass, or the like. Any insulating material may be deployed in an embodiment where the cutting blade 14 is metallic and/or electrically conductive.

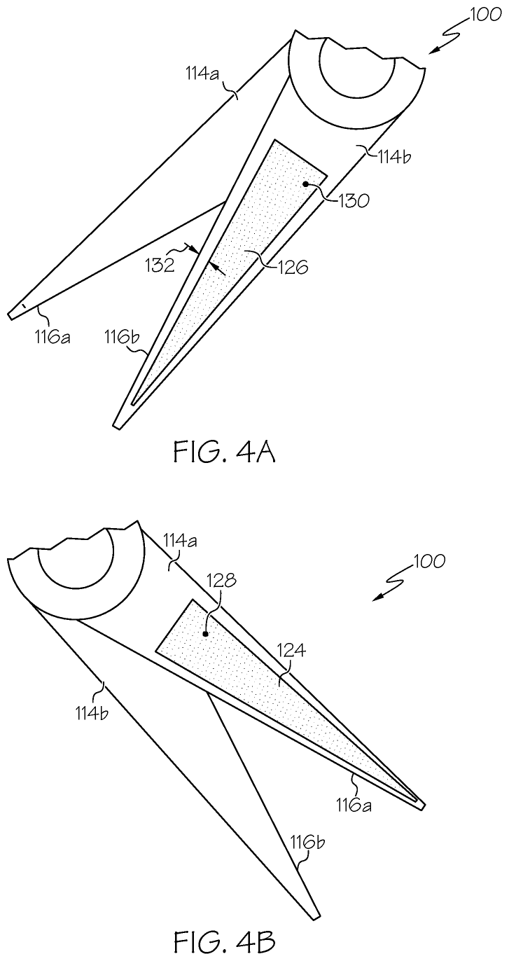

FIG. 4A depicts a perspective view of a first side of another surgical cutting and coagulating device 100, in accordance with one embodiment. FIG. 4B depicts a perspective view of a second side of the surgical cutting and coagulating device 100 of FIG. 4A, in accordance with one embodiment. The surgical cutting and coagulating device 100 takes the form of scissors. As shown, the second side (shown in FIG. 4B) of the surgical cutting and coagulating device 100 is shown with the scissors overturned relative to the first side (shown in FIG. 4A). In one embodiment, the surgical cutting and coagulating device 100 may be nonmetallic such as ceramic or zirconium and may include a first blade 114a and a second blade 114b which cooperatively operate to perform cutting. Each of the first blade 114a and the second blade 114b include respective cutting edges 116a, 116b.

Overlays 124, 126 are shown located on outer surfaces of each of the first blade 114a and the second blade 114b Like the first and second portions 24, 26 of the overlay 18 (FIG. 2A-2B) the overlays 124, 126 are sufficiently spaced from the respective cutting edges 116a, 116b via a spacing 132. The spacing 132 may be configured to provide sufficient space to allow for the cutting edges 116a, 116b to perform as conventional mechanical scissors during the incision and mechanically cut tissue. However, the spacing 132 is narrow enough to allow the overlays 124, 126 to contact the tissue during the incision. The overlays 124, 126 may have a portion bent toward the cutting edge portion 216a, 216b of the scissor blades to proximate the actual shearing edge.

The first overlay 124 of the first blade 114a includes a first electrical connection point 128 for connecting the first overlay 124 to an RF (radio frequency) generator, such as the RF generator 50. Likewise, the second overlay 126 includes a second electrical connection point 130 for connecting the second overlay 126 the RF generator.

As an incision is initiated, the overlays 124, 126 come in contact with the tissue and make an electrical connection. With voltage supplied by the RF generator through the electrical connection points 128, 130, the overlays are energized and cause current to flow into the localized tissue and impart coagulation as the mechanical cutting edges 116a, 116b cut the tissue. Like the previously described surgical cutting and coagulating device 10, the surgical cutting and coagulating device 100 provides for a bipolar cutting technique whereby only a small portion of localized tissue is subject to the electrical current.

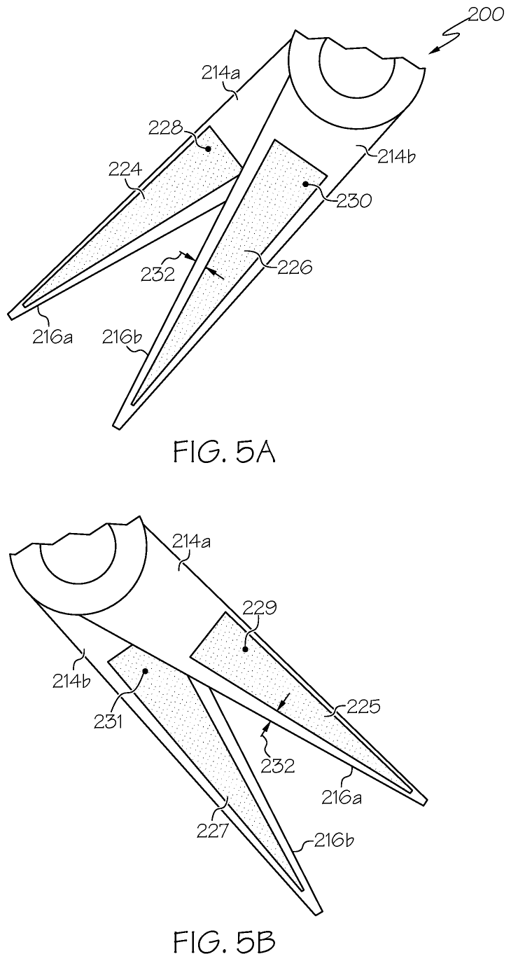

As shown, only the outer surfaces of the first and second blades 114a, 114b include the overlays 124, 126. Thus, the surgical cutting and coagulating device 100 only includes two overlays, one on each outer side of the respective first blade 114a and second blade 114b. However, in other embodiments, the overlays 124, 126 may be attached to the inner surfaces (i.e. the inner surface of the blade 114a of FIG. 4A includes the overlay 124 and the inner surface of the blade 114b of FIG. 4B includes the overlay 126). Alternatively, overlays may be attached to both surfaces of each of the blades as well, as shown in the embodiment of FIGS. 5A and 5B.

FIG. 5A depicts a perspective view of a first side of another surgical cutting and coagulating device 200, in accordance with one embodiment. FIG. 5B depicts a perspective view of a second side of the surgical cutting and coagulating device 200 of FIG. 5A, in accordance with one embodiment. The surgical cutting and coagulating device 200 may be the same as the surgical cutting and coagulating device 100 described hereinabove. As shown, the second side (shown in FIG. 5B) of the surgical cutting and coagulating device 200 is shown with the scissors overturned relative to the first side (shown in FIG. 5A). Thus, the surgical cutting and coagulating device 200 includes a first blade 214a and a second blade 214b which cooperatively operate to perform cutting. Each of the first blade 214a and the second blade 214b include respective cutting edges 216a, 216b. Overlays 224, 226 are shown located on the inner surface of the first blade 214 and the outer surface of the second blade 214b, respectively Like the overlays 124, 126, the overlays 224, 226 are sufficiently spaced from the respective cutting edges 216a, 216b via a spacing 232. Like the overlays 124, 126, the overlays 224, 226 include respective electrical connection points 228, 230 for supplying voltage from the RF generator to the overlays 224, 226.

Unlike the surgical cutting and coagulating device 100, the surgical cutting and coagulating device 200 includes overlays 225, 227 on the second side of the surgical cutting and coagulating device 200, as shown in FIG. 5B. The overlays 225, 227 may include their own respective electrical connection points 229, 231 for receiving voltage from the RF generator. Thus, the surgical cutting and coagulating device 200 includes overlays on each side of the first blade 214a and the second blade 214b. Inside surfaces of the cutting blades 214a, 214b may be recessed to accommodate overlay clearance for the overlays 224, 227. The RF Generator may be connected to energize any combination of overlays for optimal coagulation.

While not shown in FIGS. 4A-5B, it is also possible to employ an insulating layer between the cutting blades 114a, 114b, 214a, 214b and the respective overlays 124, 126, 224, 226. In such an embodiment, it is possible that the cutting blades 114a, 114b, 214a, 214b are made from a metallic material, rather than ceramic or zirconium. The insulating layer may be, for example, a ceramic insulator, a nonconductive epoxy, a glass, or the like. Any insulating material may be deployed in an embodiment where the cutting blade 114a, 114b, 214a, 214b is metallic and/or electrically conductive.

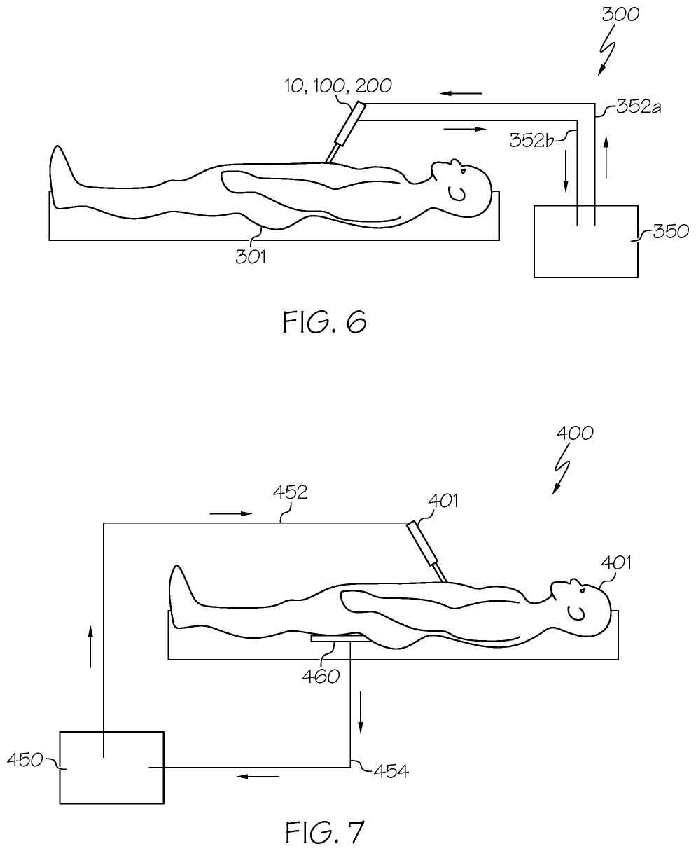

FIG. 6 depicts a schematic view of a bipolar electrosurgery system 300 deploying at least one of the surgical cutting and coagulating devices 10, 100, 200 described above in use on a patient 301, in accordance with one embodiment. As shown, current flows from a generator 350 through the wires 352a, 352b to the surgical cutting and coagulating device 10, 100, 200. The bipolar electrosurgery system 300 does not include a patient pad, and the current passes only through the tissue between the electrodes (i.e. the overlays) of the surgical cutting and coagulating devices 10, 100, 200.

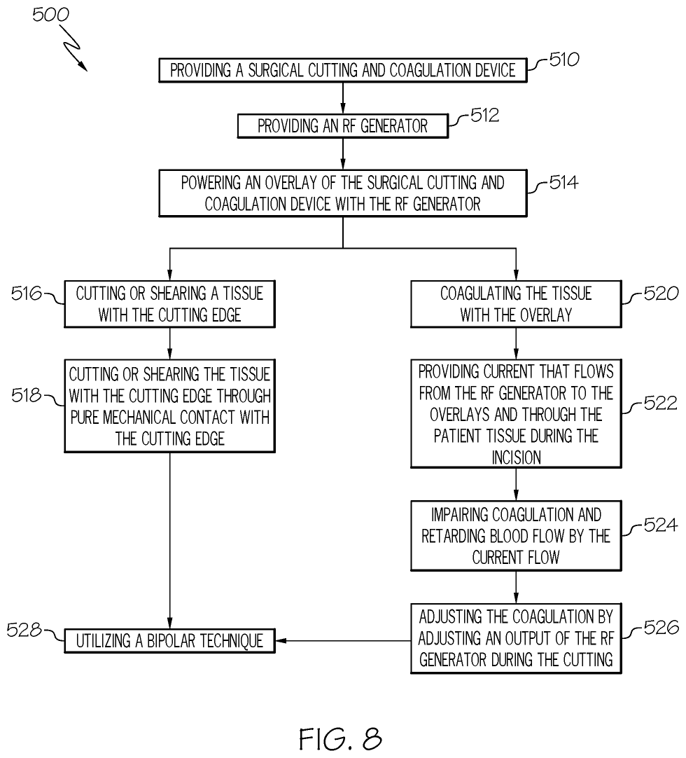

FIG. 7 depicts a schematic view of a monopolar electrosurgery system 400, in accordance with one embodiment. The monopolar electrosurgery system 400 contrasts the bipolar electrosurgery system 300 in that a patient pad 460 is deployed. A generator 450 provides current to a cutting instrument 401 and the patient pad 460 through the respective wires 452, 454. The current flows through the body of a patient 401 between the cutting instrument 401 and the patient pad 460.

FIG. 8 depicts a method 500 of surgical cutting and coagulating using a surgical cutting and coagulating device, in accordance with one embodiment. The method 500 includes a first step 510 of providing a surgical cutting and coagulation device, such as one of the devices 10, 100, 200, or the various alternatives thereof described herein above. The method 500 further includes a step 512 of providing an RF generator, such as the RF generator 50. The method 500 then includes a step 514 of powering an overlay of the surgical cutting and coagulating device with the RF generator, such as the overlays 18, 124, 126, 224, 225, 226, 227.

The method 500 includes a step 516 of cutting or shearing a tissue with a cutting edge of the surgical cutting and coagulating device, such as the cutting edge 16, 116a, 116b, 216a, 216b. This cutting or shearing the tissue with the cutting edge may be performed with pure mechanical contact with the cutting edge at a step 518.

Simultaneous to the cutting occurring in steps 516 and/or 518, the method 500 includes a step 520 of coagulating the tissue with the overlay and a step 522 of providing current that flows from the RF generator to the overlays and through the localized patient tissue during the incision. The method 500 includes a step 524 of imparting coagulation and retarding blood flow by the current flow through the overlays. The method 500 further includes a step 526 of adjusting the coagulation by adjusting the output of the RF generator during the cutting.

In the above manner, the method 500 contemplates a step 528 of utilizing a bipolar technique during the cutting and coagulating with the surgical cutting and coagulating device.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

I claim:

1. A surgical cutting and coagulation device comprising:
a cutting blade having a cutting edge; and
a metallic overlay affixed to the cutting blade configured to contact tissue during an incision of the tissue by the cutting edge of the cutting blade,
wherein the cutting blade is configured to cut or shear the tissue with the cutting edge while the metallic overlay touches and coagulates the tissue, and wherein the metallic overlay is powered by an RF generator.

2. The surgical cutting and coagulation device of claim 1, further comprising a spacing between the cutting edge and the metallic overlay, wherein the spacing is configured to allow for the cutting edge to perform as a conventional cutting device during the incision, and wherein the spacing allows the metallic overlay to contact the tissue during the incision.

3. The surgical cutting and coagulation device of claim 1, wherein the cutting edge is located on a shared perimeter of a first surface and a second surface of the cutting blade, and wherein the metallic overlay includes a first portion that is located on the first surface of the cutting blade and a second portion that is located on the second surface of the cutting blade.

4. The surgical cutting and coagulation device of claim 3, wherein the metallic overlay includes a first electrical connection point located on the first portion of the metallic overlay on the first surface for connecting the first portion of the metallic overlay to the RF generator, wherein the metallic overlay includes a second electrical connection point located on the second portion of the metallic overlay on the second surface for connecting the second portion of the metallic overlay to the RF generator.

5. The surgical cutting and coagulation device of claim 3, wherein each of the first portion and the second portion of the metallic overlay is made of a metallic material that is bonded, affixed or coated to each of the respective first and second surfaces of the cutting blade.

6. The surgical cutting and coagulation device of claim 5, wherein the cutting blade is made of ceramic or zirconium.

7. The surgical cutting and coagulation device of claim 6, wherein each of the first portion and the second portion of the metallic overlay is made of a thin sheet of metal affixed to the cutting blade.

8. The surgical cutting and coagulation device of claim 7, wherein the thin sheet of metal is a thin sheet of silver having a thickness of about 0.01 inches.

9. The surgical cutting and coagulation device of claim 6, wherein each of the first portion and the second portion of the metallic overlay is made of a conductive thick film or epoxy coated onto the cutting blade.

10. The surgical cutting and coagulating device of claim 1, further comprising an insulating layer located between the cutting blade and the metallic overlay.

11. The surgical cutting and coagulating device of claim 1, wherein RF current provided by the RF generator is configured to flow from the RF generator to the metallic overlays and through the patient tissue during the incision to impart coagulation and retard the flow of blood.

12. The surgical cutting and coagulation device of claim 1, further comprising the RF generator.

13. A surgical cutting and coagulation device comprising:
a mechanical cutting member; and
an electrical coagulation member with two or more metallic contacts,
wherein the mechanical cutting member cuts or shears tissue with a knife edge while the electrical coagulation member touches and coagulates the tissue, and wherein the coagulation member is powered by an RF generator.

14. The surgical cutting and coagulation device of claim 13, wherein the knife edge is located between a first surface and a second surface of the mechanical cutting member, and wherein the electrical coagulation member includes a first portion that is located on the first surface of the mechanical cutting member and a second portion that is located on the second surface of the mechanical cutting member.

15. The surgical cutting and coagulation device of claim 13, further comprising a spacing between the knife edge and the electrical coagulation member, wherein the spacing is configured to allow for the knife edge to perform as a conventional cutting device during the incision, and wherein the spacing allows the electrical coagulation member to contact the tissue during the incision.

16. A method of surgical cutting and coagulation comprising:
providing the surgical cutting and coagulation device of claim 1;
providing the RF generator;
powering the metallic overlay with the RF generator;
cutting or shearing a tissue with the cutting edge; and
during the cutting or shearing the tissue with the cutting edge, coagulating the tissue with the metallic overlay.

17. The method of claim 16, further comprising:
providing current, by the RF generator, that flows from the RF generator to the metallic overlays and through the patient tissue during the incision; and
imparting coagulation and retarding blood flow by the current flow.

18. The method of claim 17, further comprising utilizing a bipolar technique during the cutting.

19. The method of claim 17, further comprising adjusting the coagulation by adjusting an output of the RF generator during the cutting.

20. The method of claim 16, further comprising cutting or shearing the tissue with the cutting edge through pure mechanical contact with the cutting edge.
